(19) United States
(12) Patent Application Publication
(10) Pub. No.: US 2023/0404929 A1
(43) Pub. Date: Dec. 21, 2023

(54) Intradermal Delivery of Extracellular Vesicle-Encapsulated Curcumin Using Dissolvable Microneedle Arrays

(71) Applicant: Carnegie Mellon University, Pittsburgh, PA (US)

(72) Inventors: O. Burak Ozdoganlar, Pittsburgh, PA (US); Phil G. Campbell, Pittsburgh, PA (US); Saigopalakrishna Saileelaprasa Yerneni, Pittsburgh, PA (US); Ezgi Pinar Yalcintas, Pittsburgh, PA (US)

(73) Assignee: Carnegie Mellon University, Pittsburgh, PA (US)

(21) Appl. No.: 18/035,832

(22) PCT Filed: Dec. 1, 2021
(86) PCT No.: PCT/US21/61497

§ 371 Date: May 8, 2023

Related U.S. Application Data
(60) Provisional application No. 63/119,904, filed on Dec. 1, 2020.

(51) Int. Cl. A61K 9/50; A61K 9/00; A61K 31/12; A61K 47/42; A61K 47/38; A61K 47/26; A61M 37/00

(52) U.S. Cl. CPC A61K 9/5068; A61K 9/0021; A61K 31/12; A61K 47/42; A61K 47/38; A61K 47/26; A61M 37/0015; A61M 2037/0023; A61M 2037/0053

(57) ABSTRACT

A therapeutic delivery system uses an engineered extracellular vesicle-albumin hybrid carrier for curcumin, which is embedded in dissolvable microneedle arrays. The co-encapsulation of curcumin with albumin in extracellular vesicles extends curcumin's stability. The incorporation of therapeutic loaded carrier into microneedle arrays does not alter its cell uptake properties or bioactivity. Moreover, the bioactivity of therapeutic loaded carrier can be preserved for at least one year when encapsulated in microneedle arrays and stored under room temperature storage conditions. The microneedle arrays of the delivery system are fabricated using molding and casting processes. The extracellular vesicle carrier can be loaded using sonication.

### CROSS-REFERENCE TO RELATED APPLICATIONS

This application claims the benefit under 35 U.S.C. § 119 of U.S. Provisional Application Ser. No. 63/119,904, filed Dec. 1, 2020, which is incorporated herein by reference.

### STATEMENT REGARDING FEDERALLY SPONSORED RESEARCH

Not applicable.

### BACKGROUND OF THE INVENTION

The present disclosure is related generally to the delivery and administration of bioactive compounds. More specifically, the disclosure is related to dissolvable microneedle arrays, which can be used to deliver curcumin in vivo via a hybrid carrier comprising extracellular vesicles embedded in the microneedles of the array.

Curcumin is a natural, low molecular weight polyphenol derived from turmeric that exhibits potent anti-inflammatory, anti-cancer, and antioxidant activity. Curcumin is a potential therapeutic for various diseases and conditions, including cancer, autoimmune diseases, cystic fibrosis, Alzheimer's, and malaria. However, curcumin's low solubility, bioavailability, and stability in vivo hinder its efficacy as a clinical therapeutic. When administered orally, curcumin degrades at intestinal pH and is rapidly metabolized. Similarly, when injected systemically in blood, it degrades within minutes and undergoes rapid first-pass metabolism, resulting in a short blood-circulation half-life.

Curcumin has also shown potential benefits in topical applications for the treatment of skin diseases and wound healing. Although curcumin is a small molecule (MW=368 Da), due to its hydrophobic nature, its permeability through the cutaneous layers is poor, even when used with toxic skin permeation enhancers such as dimethylsulphoxide. Moreover, curcumin's lack of stability in the aqueous skin microenvironments and its low cellular uptake pose challenges in achieving therapeutically effective doses through topical applications. Carriers such as silica nanoparticles, poly(lactic-co-glycolic acid) nanoformulations, or cationic liposomes have been used, but have failed to address the permeability, stability, and cellular uptake challenges posed by the local application of curcumin to the skin.

Albumin has also been explored as a carrier for curcumin to improve its bioavailability and stability. Albumin is the most abundant plasma protein that has evolved for carrying biomolecular cargo, including nutrients, metabolites, hormones, and other proteins in the blood. Albumin's inherent biocompatibility, multiple binding sites, and low toxicity make it a unique drug-carrier, including for hydrophobic drugs. Albumin conjugation has been shown to increase curcumin stability and bioavailability. Yet, permeability remains an issue with curcumin/albumin compounds. In addition, delivery of the carrier remains a challenge.

Other cell-derived structures, extracellular vesicles (EVs), have been explored as carriers. Extracellular vesicles are naturally occurring sub-micrometer cell-secreted vesicles bound by a phospholipid-based bilayer membrane and decorated with several ligands and surface cargo. In the body, their primary function is intercellular transport of biomolecules from a parent cell to a recipient cell. Given their inherent ability to carry biomolecular signaling cargo, EVs are increasingly recognized as potential candidates for exogenous drug delivery. EVs have been used to deliver hydrophobic drugs, anti-cancer drugs, enzymes, and microRNA. Some works have explored using EVs as a carrier for curcumin. However, curcumin is believed to associate with the lipid-bilayer of the EV membrane, resulting in the short-term and non-uniform release of curcumin from EVs. In addition, EVs rapidly degrade outside of the body and must be stored below room temperature. Further, upon systemic administration, EVs generally tend to accumulate in the kidney, liver, and spleen, and upon oral/intramuscular administration, EVs rapidly get metabolized. As a result, depending on the route of administration in vivo, high dosages of EVs are necessary to get a therapeutic benefit that necessitates difficult, large scale production of EVs.

Microneedle arrays (MNAs) have been used as delivery vehicles. MNAs have tens to hundreds of micro-scale needles protruding from a common base. In dissolvable MNAs (dMNAs), the delivered biocargo is mixed or co-located with a water-dissolvable polymer in its gel form (e.g., carboxymethyl cellulose, trehalose, other carbohydrates). Drug-loaded dMNAs can penetrate the superficial skin layers without failure and dissolve within the skin microenvironments.

To date, only a few attempts have been made to deliver either EVs or curcumin using dMNAs. In one work, a keratin-based detachable microneedle array patch made from keratin hydrogel microneedles supported by a hyaluronic acid base was used to deliver mesenchymal stem cell-derived EVs to enhance hair growth. In another work, an EV-loaded microneedle array driven by a nitric oxide nanomotor was used for promoting Achilles tendinopathy healing. As to curcumin, both dissolvable and coated MNAs have been tested for curcumin delivery. However, these works only focused on in vitro stability of MNA-delivered curcumin. Administration to elicit a biological response was not considered and, as noted, curcumin suffers from degradation once entering the body. Another recent work used gelatin methacryloyl MNAs to deliver curcumin. Although this work included in vivo delivery of curcumin-loaded MNAs, only the biocompatibility of the gelatin methacryloyl was analyzed, and the biological activity of delivered curcumin was not evaluated.

In summary, in vivo delivery of curcumin that yields a successful biological response has not yet been demonstrated. Therefore, it would be advantageous to create a delivery system that overcomes the limitations of existing systems.

### BRIEF SUMMARY

According to embodiments of the present disclosure is a delivery system comprising a microneedle array embedded with extracellular vesicle (EV) encapsulated curcumin. The delivery system presents a synergistic approach to improve skin-targeted delivery of curcumin by combining three approaches: albumin binding, EV encapsulation, and dissolvable microneedle array (dMNA) delivery. EVs are used as carriers to address the stability and bioavailability challenges of free curcumin. Albumin is used to create additional hydrophobic binding sites to sequester curcumin, retaining it within the EVs before cell internalization, thus prolonging the bioactive lifetime of curcumin.

EVs can be loaded sequentially via mild sonication with albumin and curcumin. The cargo is then loaded into the tips of the microneedles and the tip-loaded dMNAs are then used for targeted and precise intradermal delivery of EV encapsulated curcumin-albumin (CA-EV) to the skin microenvironments. Using a micromilling/spin-casting method, dMNAs that incorporate CA-EVs (dMNA-CA-EVs) on microneedle tips are fabricated from a mixture of carboxymethyl cellulose (CMC) and trehalose as base materials.

The delivery system permits the local skin delivery of therapeutically active curcumin using a combination of EVs, albumin binding, and MNAs. The delivery system may also be used on other human tissue, including internal organs, as MNAs have shown a wide range of applications. Using the materials and methods described herein: (1) CA-EVs obtained through mild sonication show excellent stability in vitro, (2) dMNAs with the CA-EV at their tips can be fabricated reproducibly using a room-temperature process, and the fabrication process and materials do not affect CA-EVs bioactivity, (3) CA-EVs are efficiently taken up by cells and do not exhibit cytotoxicity, (4) dMNA-embedded CA-EVs exhibit storage stability for more than 12 months without the need for refrigeration, and (5) dMNA-delivered CA-EVs are effective in both suppressing and reducing skin inflammation in vivo.

### BRIEF DESCRIPTION OF THE SEVERAL VIEWS OF THE DRAWINGS

FIG. 1 shows the delivery system.

FIG. 2 is a flowchart depicting a fabrication process of the delivery system.

FIG. 3 is a diagram showing the process of loading a hybrid carrier.

FIG. 4 is a graph showing the stability of a therapeutic agent over a period of time.

FIGS. 5A-5B are images of the delivery system, with close-up views of needle tips.

FIG. 6 is a graph showing cell internalization of the delivered therapeutic.

FIG. 7 is a graph showing efficacy after storage.

FIG. 8 is a graph depicting the biological response of the delivery system in different configurations.

### DETAILED DESCRIPTION

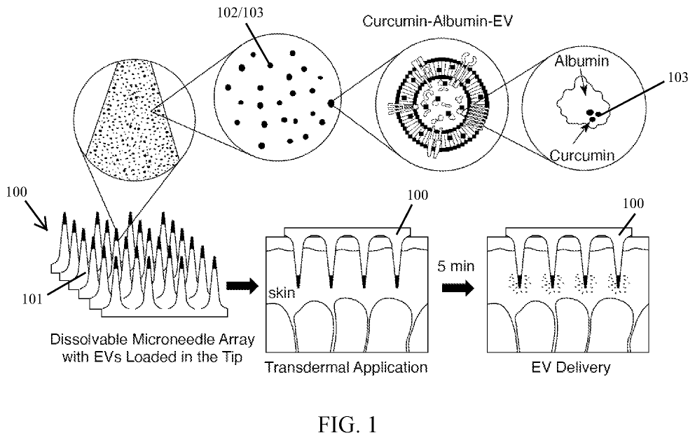

According to embodiments of the disclosure is a delivery system 100 capable of delivering therapeutics and triggering a biological response, the system 100 comprising a dissolvable microneedle array (dMNA) 101, a carrier 102, and a therapeutic agent 103. As shown in FIG. 1, which depicts one example embodiment, the carrier 102 comprises extracellular vesicles (EVs) or, alternatively, EVs combined with albumin or other materials providing attachment sites for the therapeutic agent 103. A range of therapeutic agents 103, including ones that typically show poor bioavailability, solubility, or stability using traditional delivery methods and carriers, can be delivered using the system 100. Curcumin is an example of a therapeutic agent 103 that has not realized widespread use due to poor efficacy when administered orally, intravenously, or topically. The system 100, in certain embodiments, simultaneously uses albumin binding and EV encapsulation as a hybrid carrier 102 and a dMNA 101 for the delivery of curcumin locally to the skin microenvironments and other body tissue.

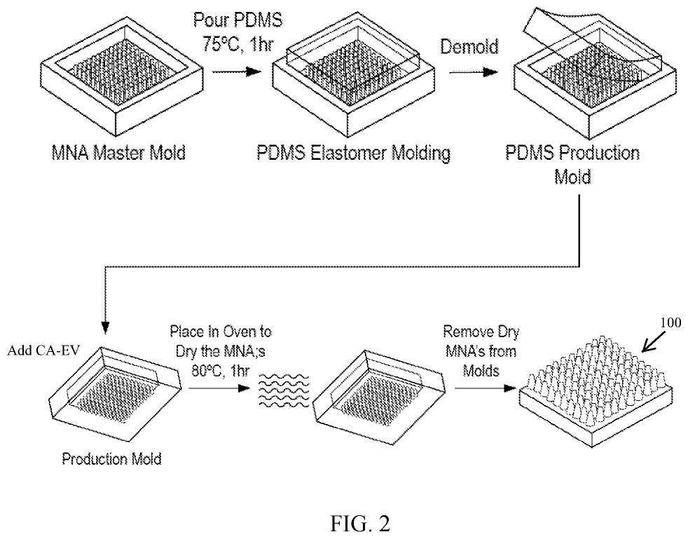

The delivery system 100 can be fabricated using molding and casting processes. One method of fabrication comprises the following steps, as shown in the flowchart depicted in FIG. 2: (1) creation of master molds from a machinable and wear-resistant material (plastic or metal) using the mechanical micromilling process; (2) fabrication of production molds from the master molds using elastomer molding; and (3) fabrication of tip-loaded dMNAs 101 from the production molds using a two-step spin-casting technique.

By way of further detail, in one example embodiment master molds are prepared by mechanical micromilling using customized micro-scale diamond tools within a high-precision computer-controlled miniature machine tool system. Next, the production molds are fabricated from a two-part elastomer, such as polydimethylsiloxane (PDMS, SYLGARD® 184 Dow Corning) with a 10:1 mixing ratio. The two-part elastomer is spread over the master mold, centrifuged for 3 mins at 2000 g, and then cured at 37° C. for 1 hour before being separated from the master molds. Next, 2.4 μl of an EV encapsulated curcumin-albumin (CA-EV) mixture is dispensed over each PDMS production mold and centrifuged for 5 minutes at 3500 g and 4° C. to fill the bottom portion of the cavities, which will form the tips of the microneedles on the dMNA 101. Finally, the structural material of dMNA 101 is prepared from a biocompatible and water-dissolvable material. In one embodiment, the material is a combination of low viscosity sodium carboxymethyl cellulose (90,000 Da, CMC, cat #C5678, Sigma-Aldrich, St Louis, MO) and D-(+)-trehalose dihydrate (Trehalose, cat #T9531, Sigma-Aldrich, St Louis, MO). A combination of carboxymethyl cellulose and trehalose strikes a balance between the mechanical stability, rapid dissolution of microneedles, and the protection of the biocargo. In addition to being biocompatible, carboxymethyl cellulose and trehalose are not cytotoxic. Additional structural or base materials for the MNA 101 may be used.

The dissolvable MNA base material is prepared by making 4% carboxymethyl cellulose and 4% trehalose in deionized water to obtain a gel-like material. The gel is then loaded into the elastomer productions molds that contain the therapeutic agent 103 loaded carrier 102 in the microneedle cavities and centrifuged for 6 hours at 10° C. and 3500 rpm to obtain the final dry dMNAs 101 incorporating CA-EVs concentrated in the needle tips, forming the delivery system 100. In one example embodiment, the shape of each microneedle in the array 101 is an obelisk with a width, height, apex angle, and fillet radius of 210 μm, 700 μm, 30°, and 35 μm, respectively. The tip-to-tip distance of microneedles is 650 μm, and each array 101 has 10×10=100 microneedles. Alternative geometries and number of microneedles can be used depending on the intended application.

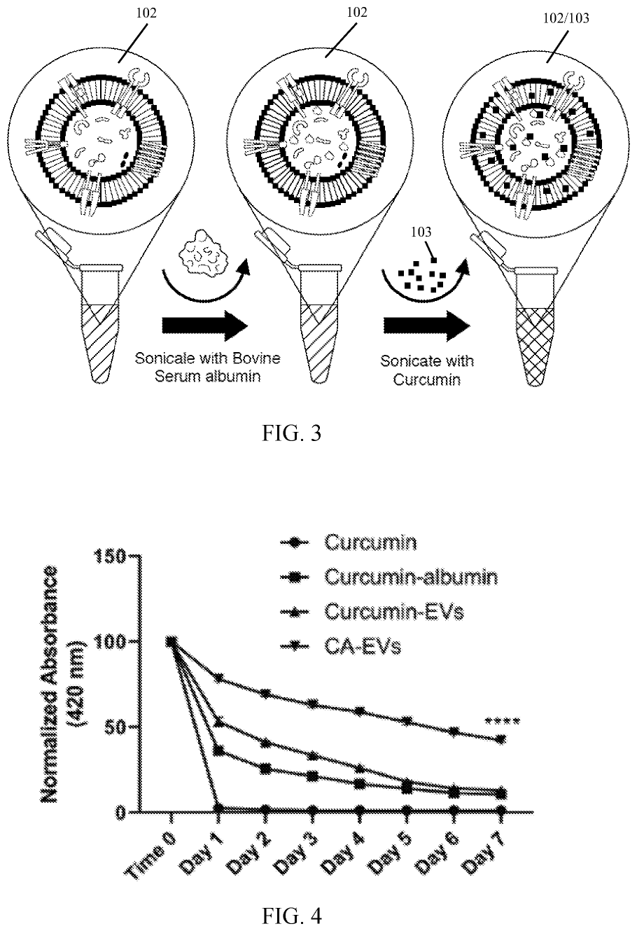

Preparation of the carrier 102, according to one embodiment, involves the concurrent use of albumin binding and EV encapsulation. To accomplish this, mild sonication is performed on the EVs, first with albumin and then with curcumin, as shown in FIG. 3. Sonication temporarily destabilizes the EV membranes, enabling albumin to enter the EV lumens. The surface-bound albumin is then removed using acid rinsing. Subsequently, albumin-encapsulating EVs are sonicated with curcumin, followed by purification using mini-size exclusion chromatography. Without the strong binding sites provided by hydrophobic domains of the loaded albumin, curcumin may exclusively associate with the EV membranes. This limits the amount of curcumin that can be delivered using EVs and may reduce the curcumin's stability. A high-performance liquid chromatography analysis of the CA-EVs obtained by this particular fabrication technique indicates that 0.56±0.01 μg of curcumin is loaded per 1 μg of albumin-EVs.

As previously described, mild sonication is used to load albumin and curcumin into EVs. To provide further detail, after adding the albumin, the albumin-EV (1 μg albumin: 1 μg of EV protein) mixture is sonicated using a 0.25″ tip at 20% amplitude, six cycles of 30 seconds on/off for 3 minutes with a 2-minute cooling period between each cycle. EV surface-bound albumin and unloaded albumin are removed by low pH acid-rinsing followed by purification of albumin-loaded EVs using the mini-size exclusion chromatography method. Following albumin loading to form a hybrid carrier 102, curcumin is loaded into the same EV-based hybrid carrier 102 using a similar sonication protocol followed by purification of curcumin-loaded EVs using mini-size exclusion chromatography.

EVs can be obtained from a variety of biological sources. The immunogenicity of EVs depends on their cellular source, and EVs derived from stem and progenitor cells and macrophages are non-immunogenic. Therefore, due to their inherent non-immunogenic properties, macrophage derived EVs can be used in the delivery system 100.

As another example, EVs can be derived from mouse cells (J774A.1-ATTC® TIB-67™) supplemented with 10% heat-inactivated fetal bovine serum (FBS; Invitrogen) and 1% penicillin-streptomycin (Invitrogen, Carlsbad, CA). EV-depleted FBS obtained by centrifugation complete media (10% FBS supplemented) at 100,000 g for 2 hours can be utilized for isolation of EVs from the cells.

Additionally, EVs from conditioned media can be isolated by size exclusion chromatography. Briefly, conditioned media is centrifuged at 2,000 g for 10 minutes at 4° C. and then at 10,000-14,000 g for 30 minutes at 4° C. The supernatant is passed through a 0.22 μm-pore Millipore filter and EVs are isolated by mini-size exclusion chromatography using 1.5 cm×12 cm mini-columns packed with 10 mL of Sepharose 2B equilibrated with phosphate-buffered saline. Given that size exclusion chromatography is a size-dependent assay, the EVs obtained using this approach contain a heterogeneous mixture, including exomeres, exosomes, and microvesicles, in size range of 30-200 nm.

Tunable resistive pulse sensing can be used to determine the size distribution of naïve, albumin-encapsulated, and curcumin-albumin EVs (i.e. therapeutic 103 loaded carrier 102). The naïve EVs had an average size of 105±34.62 nm. The EV sizes followed a Weibull-like distribution, with the large majority within 60-150 nm. Only a slight change in size distribution profile was observed after loading EVs with albumin, and nearly no size change occurred after loading albumin-EVs with curcumin. Transmission electron microscopy images indicated a heterogenous population of EVs within the measured diameter ranges. These results indicate that curcumin can be packaged as CA-EVs with relatively high efficiency and without altering the average size of the EVs.

An in vitro analysis of the stability of curcumin encapsulated within CA-EVs compares its stability to that naked, albumin-bound, and EV-encapsulated (sans-albumin) curcumin. When stored in phosphate-buffered saline at 37° C. for 3 hours, only 5% of the naked curcumin remained stable. Conjugating curcumin to albumin improved the stability to 60%, and directly loading curcumin into EVs increased the stability to 70%. On the other hand, when formed into CA-EVs, almost 90% of the curcumin remained stable for up to 3 hours.

The stability of curcumin was further analyzed for seven days in phosphate-buffered saline at 37° C. (see FIG. 4). The naked curcumin degraded entirely within one day. Only about 8% of the albumin-curcumin or EV-encapsulated curcumin remained stable up to day 7. In contrast, 45% of the curcumin in CA-EVs was stable until seven days. These results indicate that incorporating curcumin into CA-EVs increases curcumin's stability significantly, surpassing the stability of both the albumin-curcumin and EV-associated curcumin.

Extracellular vesicles have significant advantages as carriers 102 in terms of stability, bioavailability, and non-immunogenicity. Local delivery could require a low dose of EVs at the intended site, thus minimizing off-target effects and potentially avoiding large-scale production of EVs. To date, localized delivery of EVs and other extracellular vesicles remains underexplored and limited to delivery via scaffolds as solid-phase entities. However, such delivery strategies are not appropriate for delivery to the dermal microenvironment.

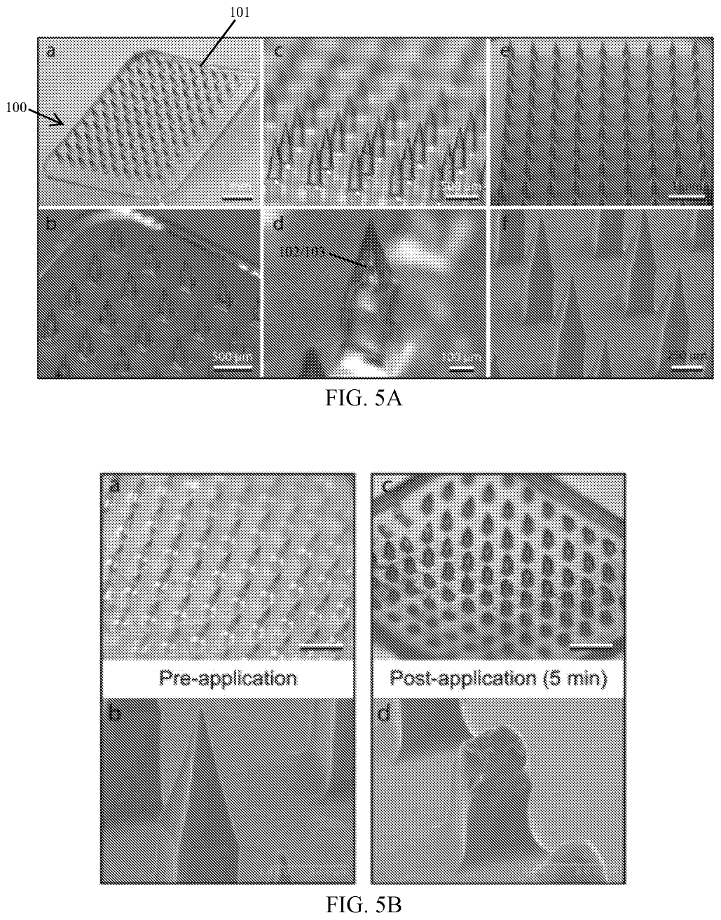

Referring to FIG. 5A, the quality of the fabricated EV-loaded dMNAs were determined using optical and scanning electron microscopy These qualitative inspections indicated that, in all inspected cases, the microneedles in an array 101 were uniform and intact. Furthermore, the optical microscopy showed that the dye-labeled EVs were successfully and uniformly loaded into the tips of the microneedles. Together, the results show that dMNAs with CA-EVs at their tips can be successfully and precisely fabricated into a delivery system 100 for certain therapeutics 103. In addition, delivery using dMNAs is painless and only causes minor skin reactions that resolve rapidly. FIG. 5B shows the tips of the microneedles 5 minutes post-application, with the dissolvable tips of the microneedles diminished.

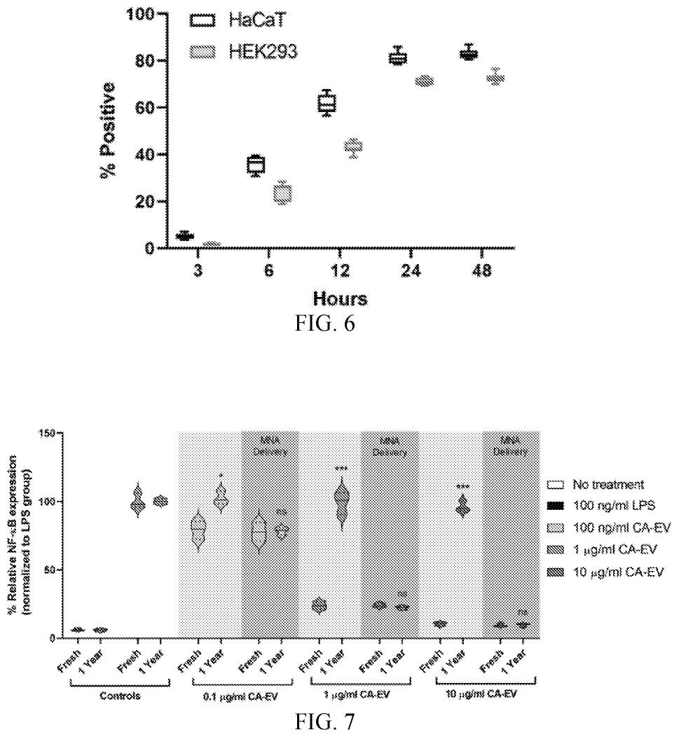

The cellular internalization of curcumin is essential for realizing its bioactivity. To evaluate the cellular uptake of curcumin, the internalization of CA-EVs is evaluated using flow cytometry and confocal microscopy. As shown in FIGS. 6, more than 75% of the targeted cells contained CA-EVs after 24 hours of incubation. Confocal microscopy was performed with PKH26-labeled EVs loaded with Alexa Fluor® 488-labeled albumin and curcumin. Confocal microscopy images at 6 hours showed co-localization of PKH26-labeled EVs and Alexa Fluor® 488-labeled albumin. These findings indicate that CA-EVs are highly efficient in cellular internalization and facilitate efficient drug delivery to cellular targets.

While cell internalization is improved with the system 100, the biological activity of CA-EVs should be preserved when embedded into the dMNAs 101 to realize the benefits of the drug delivery. The functional effectiveness of curcumin to downregulate the NF-κB transcription factor triggered by LPS induction in vitro was evaluated by utilizing NF-κB reporter RAW-Blue™ macrophage cell line. The efficacy of directly added CA-EVs with that of CA-EVs released from the dMNAs was also compared. After 24 hours of exposure to lipopolysaccharide, the NF-κB expression was measured. While unloaded EVs have no effect, the CA-EVs inhibited NF-κB upregulation in a dose-dependent manner. At 10 μg/ml, CA-EVs reduced the NF-κB expression to 18% of that induced by control LPS group. There was no statistically significant difference between direct and dMNA delivery.

The ability of CA-EVs in reversing the NF-κB levels were then evaluated. To this end, 100 ng/ml LPS was delivered to the media. After 24 hours, the indicated treatments were delivered to the media. Essentially, this study explored CA-EVs' capacity to reverse an established inflammation. At the 48-hour time point, the NF-κB levels of the RAW-Blue cells were measured. The reduction in NF-κB closely followed that seen in the inhibition analysis, including a dose-dependent reduction of NF-κB, nearly reversing the NF-κB levels to those of the negative control when 10 μg/ml CA-EVs were used, and there was no statistical difference between direct introduction or dMNA delivery of CA-EVs. These results show that dMNA fabrication and encapsulation have no impact on the biological activity of CA-EVs.

A major obstacle to clinical translation of EV-based therapeutics is their storage stability. Once isolated from biological fluids or cell culture media, EVs are susceptible to protein shedding, aggregation, and eventual degradation, especially at room temperature. Therefore, long-term (>1 month) storage of EVs commonly requires freezing them with additives such as dimethyl sulfoxide or trehalose for cryoprotection. This could pose a significant hindrance in resource-limited settings since refrigeration of biologics is not always an option in developing countries or, if available, would add to the cost of therapeutic.

The storage stability of dMNA-encapsulated CA-EVs were explored and compared to the stability of CA-EVs delivered from a stock solution. In order to evaluate the shelf life of CA-EVs, the biological activity of dMNA-encapsulated CA-EVs were evaluated and a stock solution of CA-EVs after storing them at room temperature for 12 months. As a comparison, CA-EV stock was also stored at −80° C. in phosphate-buffered saline as this is a gold standard way for long term storage of EVs. After a year, CA-EVs were analyzed and show that MNA encapsulated CA-EVs maintain their morphology, size distribution profile, and concentration and is therefore a more robust way to store EVs. Further, MNA-stored CA-EVs had intact membranes, while at −80° C. and at room temperature, CA-EVs exhibited some membrane disruption.

In evaluating the effect of storage on bioactivity of CA-EVs, incorporating CA-EVs in dMNAs dramatically improved their stability, with only a 3% (±1.46%) loss in bioactivity over the 12-months at room temperature compared to freshly made dMNA-encapsulated CA-EVs. In contrast, when CA-EVs were kept in PBS at room temperature for 12 months, CA-EVs lost their biological activity almost entirely (see FIG. 7). Therefore, dMNA encapsulated EVs could potentially be used as an off-the-shelf therapeutic option with extended storage stability, especially in resource-limited settings.

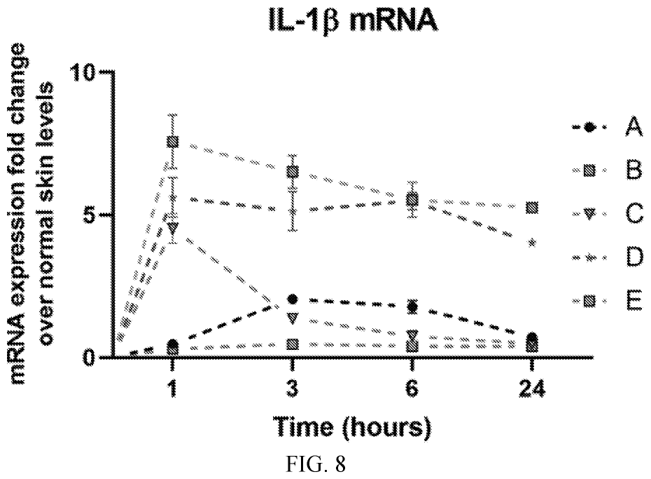

In vivo experiments were performed on a rat model to evaluate the delivery system's 100 efficacy in blocking inflammation induced by co-delivered bacterial lipopolysaccharide (LPS), thereby demonstrating functional effectiveness. Five different batches of dMNAs 101 were created with different treatments, including: (A) blank MNAs (control), (B) dMNAs containing 250 ng LPS, (C) dMNAs containing 250 ng LPS+10 CA-EVs, (D) dMNAs containing 250 ng LPS+10 μg native (non-loaded) EVs, and (E) dMNAs containing 10 μg CA-EVs. Analysis was done at 1, 3, 6, and 24 hours and inflammatory proteins measured. As shown in FIG. 8, there is a marked increase in the mRNA levels for a key inflammation marker in groups that had LPS which subsequently were down regulated in presence of CA-EVs. The LPS alone group retained high levels of inflammatory protein transcripts up to 24 hours. With CA-EVs, all three transcripts were down to normal skin levels within 24 hours of dMNA application.

These results represent the regulation of the measured inflammatory proteins due to the inhibition and resolution of inflammation resulting from CA-EV delivery. Although the cytokine levels in all groups diminished in time due to their natural regulation after resolution of inflammation, the downregulation observed with the CA-EV was rapid and steady, with most inflammatory proteins reaching the healthy-skin cytokine levels within 6 hours. Overall, these data demonstrate that the curcumin encapsulated in a EV/albumin carrier 102 and delivered by a dMNA 101 help suppresses the LPS-triggered inflammation in vivo and downregulate inflammatory proteins to resolve the inflammation entirely in less than 24 hours.

In a second in vivo demonstration, a psoriasis-like inflammation was established by applying imiquimod to mice skin. The efficacy of dMNA-delivered CA-EVs in simultaneously blocking and reversing inflammation was studied. The treatments included topical, intradermal, and dMNA application of CA-EVs, naïve EVs, and naked curcumin. The inflammation caused by imiquimod was observed to be very significant as compared to the normal skin. “Blank” and naïve EV treatments did not provide a therapeutic effect, and naked curcumin or topically applied CA-EVs only have a negligible effect. On the other hand, dMNA-delivered CA-EVs have the most significant effect in reversing the inflammation, resulting in a skin cross-section indistinguishable from that of normal skin.

When used in this specification and claims, the terms “comprises” and “comprising” and variations thereof mean that the specified features, steps or integers are included. The terms are not to be interpreted to exclude the presence of other features, steps or components.

The invention may also broadly consist in the parts, elements, steps, examples and/or features referred to or indicated in the specification individually or collectively in any and all combinations of two or more said parts, elements, steps, examples and/or features. In particular, one or more features in any of the embodiments described herein may be combined with one or more features from any other embodiment(s) described herein.

Protection may be sought for any features disclosed in any one or more published documents referenced herein in combination with the present disclosure. Although certain example embodiments of the invention have been described, the scope of the appended claims is not intended to be limited solely to these embodiments. The claims are to be construed literally, purposively, and/or to encompass equivalents.

What is claimed is:

1. A therapeutic delivery system comprising:
a microneedle array comprising a dissolvable base material;
a carrier comprising extracellular vesicles,
wherein the carrier is embedded within base material of the microneedle array.

2. The delivery system of claim 1, further comprising:
a therapeutic agent associated with the carrier.

3. The delivery system of claim 2, wherein the therapeutic agent comprises curcumin.

4. The delivery system of claim 2, wherein the extracellular vesicles encapsulate the therapeutic agent.

5. The delivery system of claim 1, wherein the carrier further comprises albumin disposed within the membrane of the extracellular vesicle.

6. The delivery system of claim 2, further comprising:
a therapeutic agent associated with the carrier.

7. The delivery system of claim 6, wherein the therapeutic agent comprises curcumin and is bound to the albumin.

8. The delivery system of claim 6, wherein the extracellular vesicles encapsulate the therapeutic agent.

9. The delivery system of any of the preceding claims, wherein the dissolvable microneedle array comprises at least one of trehalose dihydrate and carboxy methylcellulose.

10. A method of fabricating a therapeutic delivery system comprising:
forming a mold of a microneedle array;
adding extracellular vesicles to the mold and centrifuging to disperse the extracellular vesicles to within the cavities of the mold;
adding a base material to embed the extracellular vesicles within the mold; and
drying the mold to form a microneedle array containing extracellular vesicles disposed within a tip of each microneedle of the microneedle array.

11. The method of claim 10, further comprising:
adding a therapeutic agent to the extracellular vesicles through sonication prior to adding to the mold.

12. The method of claim 10, further comprising:
forming a carrier by adding albumin to the extracellular vesicles through sonication prior to adding to the mold.

13. The method of claim 10, wherein forming a mold of a microneedle array comprises:
forming a master mold via micromilling, wherein the master mold has a shape of the microneedle array;
forming a production mold by casting a polymer over the mater mold, wherein the production mold is a negative-shape of the microneedle array.

14. The method of claim 12, further comprising:
purifying the carrier using mini-size exclusion chromatography.

15. The method of claim 12, further comprising:
acid rinsing the carrier after sonication.
